(19) United States
(12) Patent Application Publication
(10) Pub. No.: US 2005/0137658 A1
(43) Pub. Date: Jun. 23, 2005

(54) Treatment of superficial pigmented and vascular lesions of the skin

(76) Inventor: David R. Hennings, Roseville, CA (US)

Correspondence Address: Ray K. Shahani, Esq., Twin Oaks Office Plaza, 477 Ninth Avenue, Suite 112, San Mateo, CA, 94402-1854, US

(21) Appl. No.: 10/738,384

(22) Filed: Dec. 17, 2003

(51) Int. Cl. A61F 7/00

(52) U.S. Cl. 607/96

(57) ABSTRACT

A method and device to treat superficial lesions in the skin such as solar lentigines that involves a light based energy delivery system in conjunction with a pulsed cooling device to selectively heat surface targets without damage to deeper skin structures.

### FIELD OF THE INVENTION

The present invention is related to therapeutic applications for light energy, and in particular, a method and device to treat superficial lesions in the skin such as solar lentigines that involves a light based energy delivery system in conjunction with a pulsed cooling device to selectively heat surface targets without damage to deeper skin structures.

### BACKGROUND OF THE INVENTION

This invention is an improvement over prior methods of selectively treating a structure in the skin without damage to adjacent tissue. Methods taught in the prior art use wavelength, pulse duration and cooling designed to protect, not treat the epidermis. Methods taught in the prior art using contact cooling to cool the epidermis and any target superficial lesions are less effective because the cooling is simultaneous with the delivery of heat or light energy resulting in a cancellation of the effect of both on the epidermis.

Solar lentigines of the skin are flat brownish pigmented spots on the skin due to increased deposition of melanin and an increased number of melanocytes. They are treated by heating with a laser or white light source until they slough off. When treated with the proper amount of energy the brown spots darken or turn gray immediately after treatment. They then blister slightly and fall off after 1 or more days. Sometimes the lesion needs several treatments. It is most difficult to treat brown spots in patients with natural pigment in their skin. These type 3 to type 5 patients may blister or show undesirable pigmentation changes after treatment because of the difficulty in avoiding damage to the slightly deeper natural skin pigment.

The current methods are non-specific with respect to wavelength absorption and depth and can cause damage to surrounding tissue and natural skin pigment. Current methods of treating lentigines utilize optical sources that emit wavelengths of 550 nm to 800 nm and use continuous or contact cooling. Blood absorbs in this region interfering with the target interaction. The wavelengths used in current art penetrate very deeply affecting the natural skin pigment, and continuous or contact cooling lowers the temperature of the target lesion so that excessive energy is needed to raise the lesions temperature up to the damage temperature of about 85 degrees C.

U.S. Pat. No. 5,405,368 to Eckhouse teaches the use of a white light flashlamp to treat skin disorders. Eckhouse does not teach selection of wavelengths specifically to target pigment, nor does Eckhouse teach selection of wavelengths specific or specifically to scatter highly and not penetrate deeply into tissue. Eckhouse does not teach selection of wavelength based on light-scattering properties, nor does Eckhouse teach removal of the contact cooling prior to treatment in order to avoid simultaneous cooling and heating of superficial lesions.

U.S. Pat. No. 5,344,418 to Gaffari and 5,814,040 to Nelson teach dynamic cooling immediately prior to a treatment pulse in order to cool only the epidermis and not penetrate deeply. These patents do not teach cooling target tissue, target structures or skin tissue a long time prior to the treatment pulse and then removing the cooling so that the cold is allowed to penetrate deep into the tissue and then treating with an energy source in such a manner that only the surface is affected. It will be understood that removal of heat is required for penetration of the cold temperature zone into and through the dermis, epidermis and subdermal tissue and structures.

### SUMMARY AND ADVANTAGES OF THE PRESENT INVENTION

It is the intent of this invention to treat superficial lesions in a more specific manner providing a safer and improved treatment.

The selection of treatment wavelength is based on the optical absorption curves of melanin and hemoglobin, and the physical properties of light scattering in the skin.

Cooling the skin is accomplished in a pulsed manner to allow for heat and cold transport to take place in a controlled and predictable manner to selectively target specific lesions with heat or optical energy that converts to heat when absorbed.

Cooling is accomplished with a pulsed cryogen spray that can be timed to the delivery of optical or heat energy in such a way that the cold can penetrate deeply into the tissue.

Optical or heat energy is delivered to the target lesion in a short pulse so that the absorbed energy does not diffuse out of the lesion but rather raises the lesion temperature until it is no longer viable.

Optical energy is delivered to the skin in a short enough wavelengths so that the natural scattering of the skin limits the penetration depth.

Optical energy is delivered to the pigmented lesions of the skin in a wavelength region that is highly absorbed by melanin but less absorbed by other components of the skin such as blood and water.

Optical energy is delivered to vascular lesions of the skin in a wavelength region that is highly absorbed by blood and less so by melanin and water.

Optical energy is generated by a laser or a white light device such as a flashlamp, medium pressure arc lamp or a filament lamp. Wavelength selection of the white light spectrum of a flashlamp is accomplished through the use of optical absorbing and reflecting filters and by optimizing the blackbody peak wavelength as taught in pending U.S. Ser. No. 10/351,273 filed Jan. 24, 2003 entitled “Method and Apparatus for Treating Skin Disorders Using a Near Black Body Flashlamp Source”, which is hereby incorporated by reference in its entirety into the present application.

### BRIEF DESCRIPTION OF THE DRAWINGS

The invention is illustrated below and represented schematically in the following drawings:

FIG. 1 shows absorption curves for melanin, hemoglobin and water.

FIG. 2 shows the reduced scattering coefficient for skin and various calculated values for collagen tissue plotted as a function of wavelength.

FIG. 3 shows a cross section of the skin with superficial pigmented and vascular lesions identified.

FIG. 4 shows a timing and temperature diagram that illustrates how the method is able to treat superficial lesions while protecting deeper structures.

FIG. 5 shows how the blackbody temperature of a flashlamp can be optimized for maximum output in the desires wavelength range.

FIG. 6 shows how reflecting and absorbing filters can be used in front of a flashlamp to select the desired wavelength range.

FIG. 7 shows a device that will accomplish the method.

FIG. 8 shows a handpiece for the device.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

Description of Near Black Body Operation

The present invention incorporates by reference U.S. Pat. No.6,117,335 and No. 6,200,466 disclosing a flashlamp system operating in the near black body regime to generate UV light for the purpose of decontaminating water. The relevant discussion of the physics of flashlamps contained therein is incorporated here by reference.

1) Flashlamp Spectra.

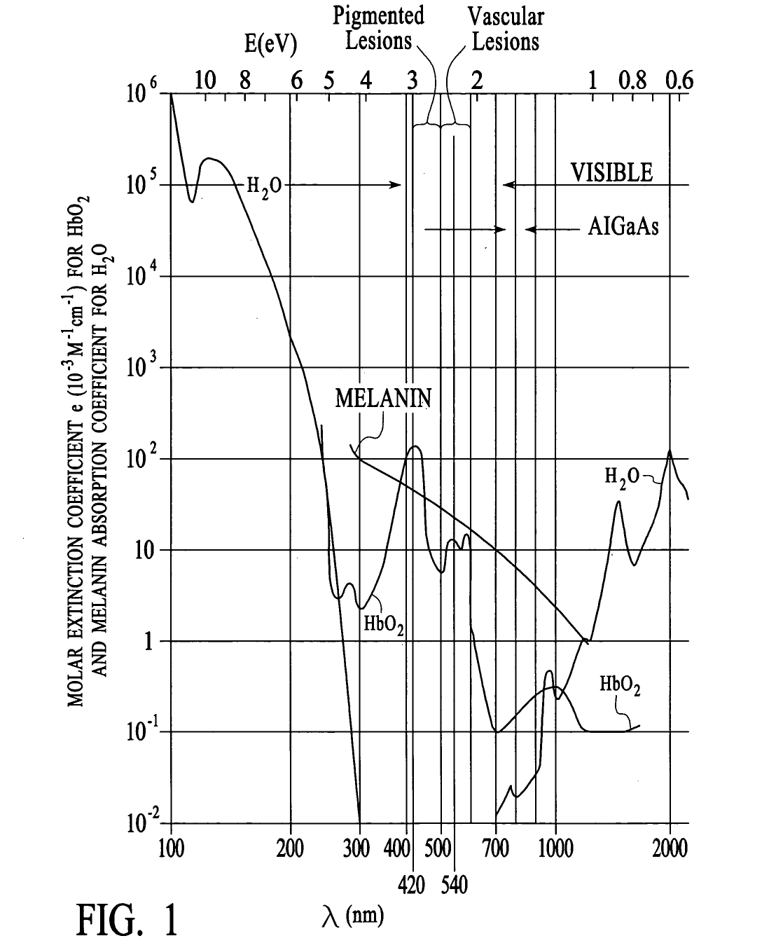

A continuum mode of radiation is created by strongly ionizing the gas within the flashlamp. This continuum radiation approaches a high-emmisivity blackbody radiation profile with increasing flashlamp power density. Illustrated in FIG. 1 are spectra for a xenon-filled flashlamp at three different levels of power density (see ILC Technology, Inc., Technical Bulletin No. 2, FIG. VIII). Power density is defined as:
P₀=(Eₒ/tₚAₛ)(watt/cm²) (1)
where: Eₒ=lamp discharge energy (joules);
tₚ=pulse duration FWHM (seconds); and
Aₛ=lamp bore surface area (cm²).

At the lowest power density of about 4 kW/cm², it is evident that the emission contains sharp peaks (line spectra) superposed on a low continuous background. Line spectra are the result of the electrons in the discharge current colliding with atoms or ions, causing within them internal transitions between bound energy levels. In relaxing from these excited states, the atoms or ions emit light energy at discrete wavelengths (bound-bound transitions), or line spectra. As the power density is increased to 14 kW/cm², the proportion of continuous background increases. The continous spectrum is generated by the deceleration of electrons in collisions with ions (Bremstrahlung) and by collisions that ionize (bound-free transitions), both of which occur more frequently at higher power densities. At the highest power density of about 70 kW/cm², the envelope of the continuum spectra approachs the reference curve, which is superposed to show the spectral distribution of an ideal black body radiator at 9500° K absolute temperature. The peak of the continuous spectrum also lies in the UV (at a wavelength less than 400 nm) and the continuous spectrum fully dominates the line spectra. These are the charactersitics of the “near black-body” state, where the photorns, and charged and neutral particles of the discharge plasma near thermal equilibrium. The approach is gradual with increasing power density; there is no abrupt demarcation point. However, conventional rules of thumb for operation of a flashlamp as a near black-body radiator are that the power density exceeds 25 kW/cm², or that the plasma temperature reaches 9500° K.

2) Flashlamp Lifetime

The flashlamp must be optimized to deliver the maximum amount of useful energy with good conversion efficiency while still maintaining a useful long lifetime. Driving the lamp harder, while producing more UV, shortens the lamp life considerably. This tradeoff must be balanced by careful consideration of pulse duration and energy input.

To maintain reasonable lamp life, the input energy to the flashlamp must be kept below 18% of the theoretical single-shot explosion energy limit. Various models are used to predict lamp life. For a lamp that is driven hard, the expected failure mode is the limit imposed by envelope material tensile stress, seal strength, and wall ablation and cracking. Then the following formulas show how the explosion energy is related to the lamp geometry, envelope material, input energy, and pulse duration (see ILC Tech. Bulletin 2).

From the dimensions and envelope material of the flashlamp, an explosion-energy constant (Kₑ) is obtained:
Kₑ=f(d)ld (2)
where:
f(d)=silica power function based on material transparency, thermal conductivity, wall thickness, and bore diameter, W sec^{1/2} cm⁻²,
l=discharge length of the flashlamp, cm,
d =bore diameter of the flashlamp, cm.
   The single-shot explosion energy Eₓ then is:
   Eₓ=Kₑ(tₚ/2)^{1/2}. (3)
   The lamp lifetime LT, in number of shots, is approximated by:
   LT=[Eₒ/Eₓ]^{-b} (4)
   where the flashlamp input energy Eₒ is in Joules, and the constant b depends on bore diameter and wall thickness. For the small bore lamps of 6 mm diameter or less (appropriate for mounting in a handheld device for dermatologic treatments) the constants to be used in Eq. (4) are Kₑ=24600, and b=8.5. To be conservative in the case of a lamp used in a commercial product, the number of shots predicted by Eq. (4) is frequently reduced by some safety factor such as 10⁻³.
   3) Advantages of a Near Blackbody Source in the Treatment of Skin Disorders

An ideal black-body emitter of a given temperature emits more energy than any real source with the same surface temperature. A flashlamp driven to near-blackbody operation thus approaches theoretical limits, and is a high power emitter providing light energy over broad spectrum. This makes possible treatments with a single source for a wide range of skin disorders. In the phototherapy treatment of skin disease, various wavelength bands are used:

| Skin Condition | Wavelengths of Treatment | |
|---|---|---|
| psoriasis | 297-320 | nm |
| vitiligo | 297-320 | nm |
| acne | 405-420 | nm |
| hair removal | 640-1200 | nm |
| reduction of vascularization | 640-1200 | nm |
| roseacea | 640-1200 | nm |

The continuous nature of near blackbody radiation allows any of these wavelengths to be made available by spectral filtering in the delivery system, to pass the desired wavelengths, and reject the unwanted bands. Additionally, the peak wavelength of the blackbody spectral distribution can be tuned (by the control of the ratio Eₒ/tₚAₛ) to weight the spectrum of the lamp for more output in the desired bands to make the lamp more efficient for a given application. Finally, the established nature of the life limits in this mode of operation permits a rational tradeoff to be made between lamp emittance (also called radiant exitance), spectral distribution, and lifetime.

4) Adjusting the Near-Blackbody Spectrum

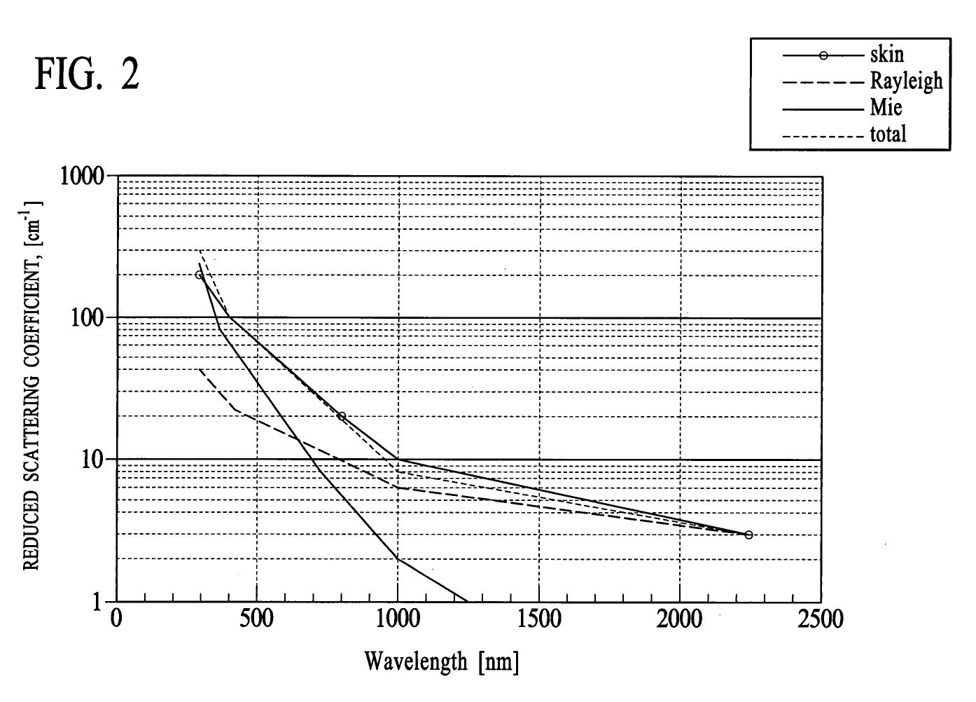

The wavelength of the peak of the blackbody spectral distribution shifts with the surface temperature T of the emitter according to Wein's Displacement Law:
lₚₑₐₖ=2898/T (5)
where lₚₑₐₖ is in micrometers, and T is the absolute temperature, in Kelvins. FIG. 2 shows this peak wavelength shift for three different blackbody temperatures in the range of operation of the device of the present invention. The wavelength of the peak of the curve moves from 366 nm for the lowest curve at temperature of 7911° K, to 333 nm for the middle curve at 8703° K, and to 300 nm for the upper one at 9660° K.

For the change from the lowest to the highest temperature, the maximum emittance per unit wavelength interval (relative intensity) rises by a factor of 2.7, in accordance with Plank's Radiation Formula (see U.S. Pat. No. 6,117,335, Eq. (2)). When this formula is integrated over all emitting wavelengths, the Stefan-Boltzmann Law results, which states that the total power emitted over all wavelengths, per cm² of blackbody surface area, increases with the fourth power of the surface's absolute temperature:
P=sT⁴. (6)
The proportionality constant is called Stefan's constant and is equal to:
s=5.67×10⁻¹² W cm⁻²(° K)⁻⁴.

The power emitted from the flashlamp can be estimated as the input energy E ₒ times the average radiation efficiency (0.85) to get the total radiation, divided by the average time over which this energy is delivered (the pulse length tₚ). Balancing this power against the that from the Stefan-Boltzmann law gives the equivalent blackbody temperature of the lamp, T_{BB}, the temperature of a perfect blackbody emitting over the same area as the lamp at the same total power:
AₛsT_{BB}⁴=(Total Power)=(0.85)(Eₒ/tₚ)
or
T_{BB}=[(0.85)(Eₒ/stₚAₛ)]^{1/4}. (7)
The temperature given by Eq. (7) is an upper bound for the plasma temperature of the lamp, since to the degree that a portion of the output spectrum may still exist as line spectra, there is less power to be dissipated as blackbody radiation and a slightly lower plasma temperature may result.

Eq.(7) shows directly why the lamp spectrum moves towards the continuous blackbody spectrum as the lamp power density Eₒ/tₚAₛ of Eq. (1) is increased to balance the increasing power density, the average energy (temperature) of the particles in the plasma must increase to radiate more, and the thermal interactions swamp the competing means of radiating.

In general, to achieve a higher plasma temperature to increase the energy radiated into a desired bandwidth, or to increase the overall output energy, the application of shorter pulses of electrical input energy will be useful. Flashlamps are often driven with pulse forming networks (PFN's) where the input energy is determined by the charge on the capacitance C, and the time to deliver charge to the lamp is determined by the inductance L. Varying L conveniently adjusts the power density delivered and thus the lamp's blackbody radiation characteristics.

The tradeoff is that the lamp life decreases with explosion energy Eₓ, which by Eq.(3) is also a function of the pulse length tₚ. Substituting Eq.(3) into Eq.(4) shows that the expected lifetime LT of the lamp scales proportionally to:
LT=constant (tₚ)^{4.25}, (8)
a rapid decrease as the pulse length decreases.
5) Optimizing T_{BB} for Dermatoloaical Applications

In the prior art patents there is presented the logic for optimizing the operating point of the flashlamp for his application of water decontamination through control of the pulse length. In dermatological applications, the situation is analogous.

For purposes of concreteness or to be more definite, consider the most demanding dermatological application, that of the phototherapy treatment to clear psoriasis, which requires application of ultraviolet light in the range of 297-320 nm. Actually, the most effective ultraviolet band is 293 nm to 309 nm band (see J. A. Parrish et. al., Journal of Investigative Dermatology, v. 76 (1981) pp. 359-362, “Action Spectrum for the Phototherapy of Psoriasis”). These wavelengths are the points on the action spectrum where the effectiveness in clearing placque drops to 10% of that at the 300 nm peak of the spectrum. However, wavelengths shorter than 297 nm lie within what is believed to be the photocarcinogenesis action spectrum for humans (though it was measured on hairless mice; see C. A. Cole, et. al., Photochemistry and Photobiology, v. 43 (1986) pp. 275-284, “An Action Spectrum for UV Photocarcinogenesis”). Thus the repeated exposure to significant energy at wavelengths shorter than this will eventually cause skin cancer. Psoriasis sufferers generally accept this small risk, where the spectra overlap, to be cleared of the effects of their disease.

Thus both applications have a short wavelength limit, below which the lamp output is not useful. In the prior art case, this was the transparency limit of the lamp envelope, about 185 nm. In the case of psoriasis treatment, the lower limit is the turn-on of the carcinogenesis spectrum at 297 nm. Logic of the prior art is to adjust the pulse length, to shift the peak wavelength of the blackbody spectral distribution just to the long-wavelength side of the useful short wavelength limit. He showed that in generating light in the useful band for the water decontamination application (which extended from 185 nm up to 400 nm) that the efficiency did not depend strongly on T_{BB} as long as the blackbody peak wavelength, and the useful short wavelength limit were close. Essentially, driving the lamp harder at this point, to move the spectrum down to shorter wavelengths, generated more light that fell in wavelength below the short wavelength limit, with a severe penalty in lamp lifetime. Logic used in the prior art determines an optimum operating point-position the blackbody spectral peak just to the right of the short wavelength limit at the first acceptable value for lamp lifetime. For the psoriasis application, the same logic gives the middle curve.

Actually, in the psoriasis application the optimum blackbody peak moves further to the right when it is considered that it is often an advantage to use a greater number of lower energy shots, to give adequate resolution in dosage control by counting the number of shots. The lamp life considerations favor this approach.

For example, five shots to complete a dose gives a 20% dosage control with ±1 shot added or dropped. This is about what is desired. Consider then the alternatives of the two operating points represented by the two lower curves From the Stefan-Boltzmann law (Eq. (6)), the power densities for the two lower curves are in the ratio of 1:1.46. Holding the input energy to the lamp constant, reaching these two operating points would then require pulse lengths in the ratio of 1:1/1.46. By the scaling law Eq.(8), the shorter pulse length would reduce the lamp lifetime by a factor of 1/5.06=0.2=[1/1.46]^{4.25}. The useful energy per shot with the shorter pulse length is only 46% larger, or the total dose with the lower curve can be reached in 7 shots, if it were reached in 5 shots with the middle curve as operating point. Using 46% more shots, with 5 times as many shots available from the lamp, is a better tradeoff if the resultant treatment times are acceptable to the doctor and patient.

Additional pulsed blackbody, deep-UV radiators are described in U.S. Pat. Nos. 5,789,755 and 6,028,316, both entitled METHOD AND APPARATUS FOR REMOVAL OF MATERIAL UTILIZING NEAR-BLACKBODY RADIATOR MEANS, both of which are hereby expressly incorporated by reference in their entireties herein.

Additional pulsed blackbody, deep-UV water purification systems are described in U.S. Pat. Nos. 6,117,335 and 6,200,466, both entitled DECONTAMINATION OF WATER BY PHOTOLYTIC OXIDATION/REDUCTION UTILIZING NEAR BLACKBODY RADIATION, both of which are hereby expressly incorporated by reference in their entireties herein.

Treatment of Superficial Pigmented and Vascular Lesions of the Skin

FIG. 1 shows absorption curves for melanin, hemoglobin and water. The preferred wavelength region to treat melanin without impacting blood is in the region of 440 nm to about 520 nm as this region shows the melanin absorption curve to be substantially higher that the blood absorption curve.

FIG. 2 shows the reduced scattering coefficient for skin and various calculated values for collagen tissue plotted as a function of wavelength. Wavelengths below 440 nm are not used because of potential toxic effects of WVA on the skin. Wavelengths above 600 nm are not preferred because they are scattered poorly in the skin causing deep penetration.

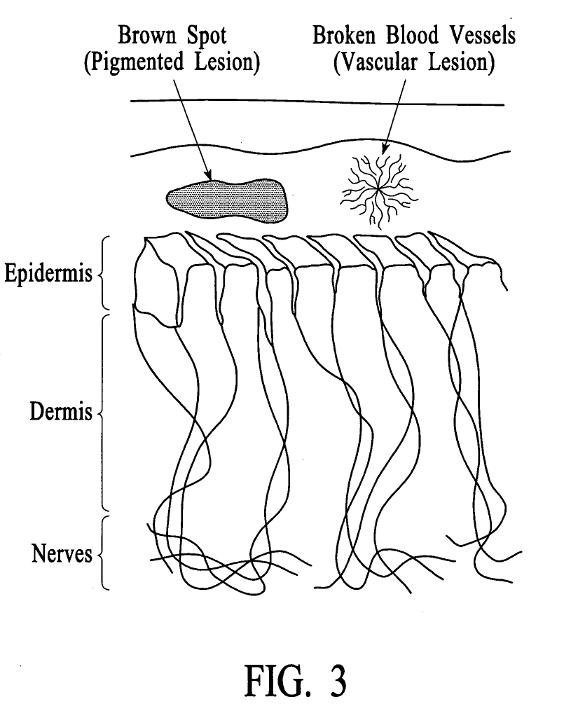

FIG. 3 shows a cross section of the skin with superficial pigmented and vascular lesions identified. These target lesions are close to the skin surface. Structures that need protection include natural skin pigmentation, deeper vascular structures and nerve fibers, and others.

Optical Pulse lengths of 10 to 50 msec are used to limit the heat conduction into the skin yet are sufficient to treat the target lesion as taught by Anderson.

Pulsed Cooling durations of 20 to 500 msec are used long in advance of the treatment energy to allow the cold to go deeply into the skin and not counteract the surface effects of the optical energy.

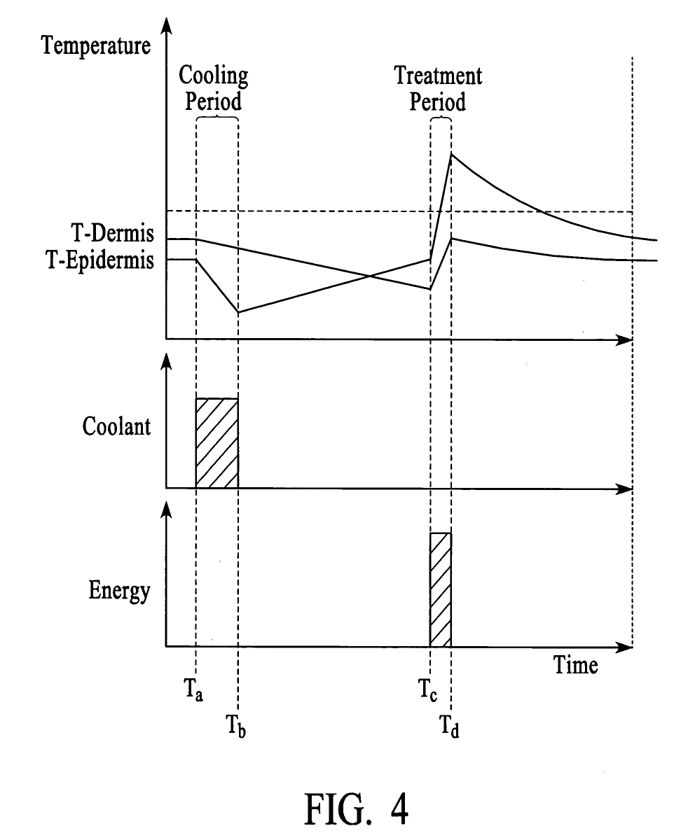

FIG. 4 shows a timing and temperature diagram that illustrates how the method is able to treat superficial lesions while protecting deeper structures. As shown, the coolant is delivered such as in a spurt of cryogenic material directly to the surface of the skin, prior to delivery of the treatment energy furthermore, a predetermined time period is allowed between the termination of the coolant and initiation of the treatment energy.

Cooling is initiated at time Tₐ, and the particular spurt of cooling is terminated at time T_{b}. The time period between Tₐ and T_{b} comprise the period of cooling. The time period between T_{b} and T_{c} comprise the period of delay between the cooling period and the treatment period. Delivery of treatment energy is initiated at time T_{c}, and delivery of treatment energy is terminated at time T_{d}. The time period between T_{c} and T_{d} comprise the treatment period.

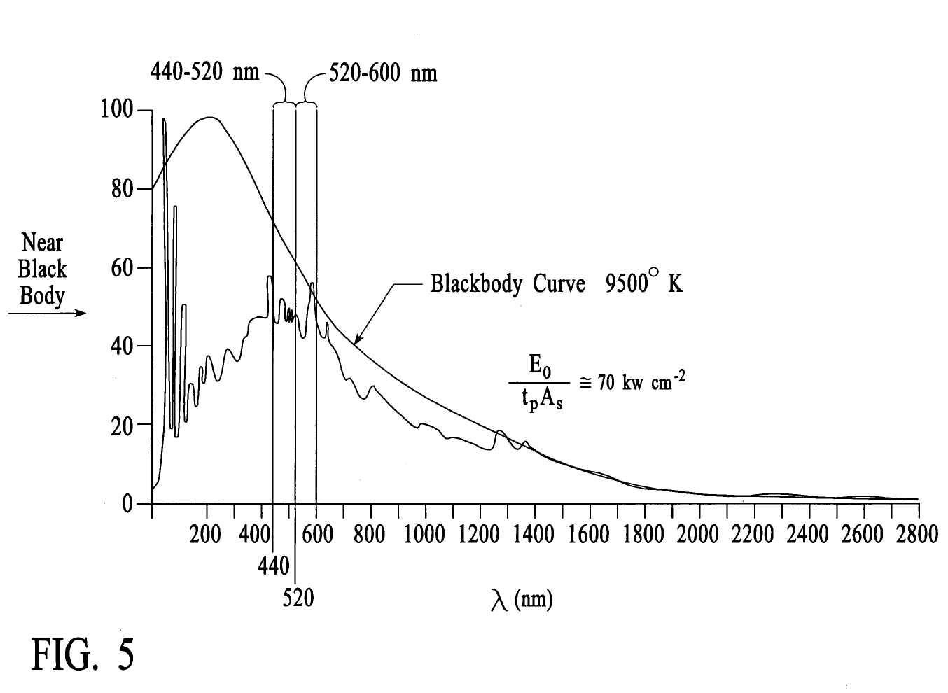

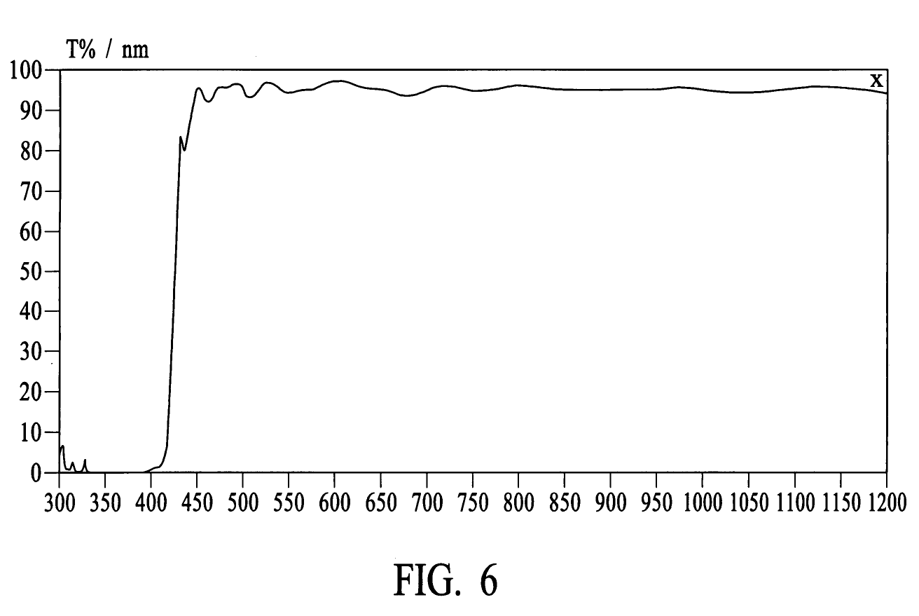

FIG. 5 shows how the blackbody temperature of a flashlamp can be optimized for maximum output in the desires wavelength range. FIG. 6 shows how reflecting and absorbing filters can be used in front of a flashlamp to select the desired wavelength range.

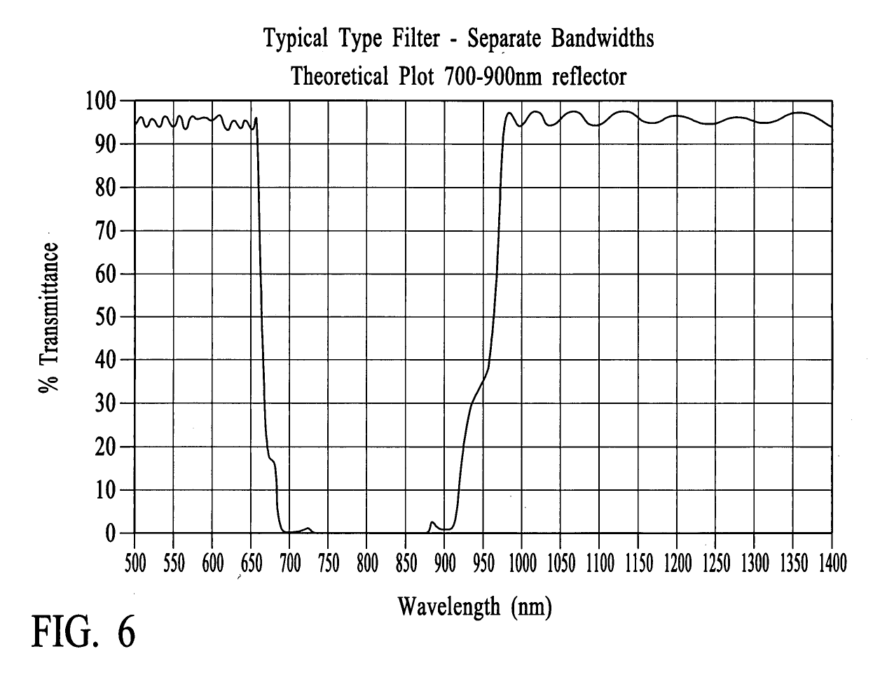

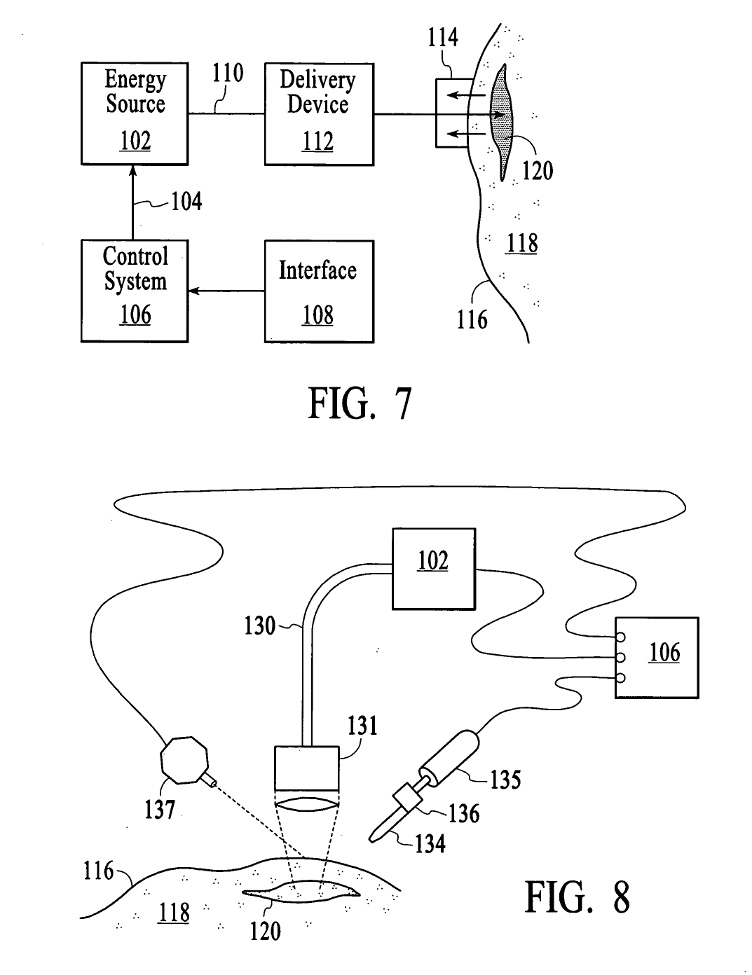

FIG. 7 shows a device that will accomplish the method. FIG. 8 shows a handpiece for the device. Superficial vascular lesions such as Rosacea involve flushing and red coloration due to dilation of capillaries in the skin of the nose, forehead and cheeks. Treatment of these lesions is similar to pigmented lesions except that a different wavelength range is used. The goal with vascular lesions is to use wavelengths that are highly absorbed by blood and less so by melanin. This region is from 520 nm to 600 nm. Wavelengths shorter than 520 nm have too much melanin absorption. Wavelengths longer than 600 nm penetrate too deeply to be optimum for superficial lesions. Relatively short treatment pulses of 10 to 50 msec are used to limit heat conduction to deeper tissue. Relatively long cooling durations of 20 to 500 msec are used to enhance conduction into deeper tissues. A delay between cooling and treatment of 100 to 5000 msec is necessary in this invention to allow the cooling to penetrate deeply into tissue.

Unless defined otherwise, all technical and scientific terms used herein have the same meaning as commonly understood by one of ordinary skill in the art to which this invention belongs. Although any methods and materials similar or equivalent to those described can be used in the practice or testing of the present invention, the preferred methods and materials are now described. All publications and patent documents referenced in this application are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

1. A method of treating superficial pigmented lesions comprising the following steps:
A. Generating pulsed energy from an energy source for treatment of the target tissue or structures;
B. Cooling the surface of the skin through delivery of at least one pulse of cryogenic material to the surface of the skin, the at least one pulse of cryogenic material initiated prior to delivery of the pulsed treatment energy to cool deep into the target tissue or structures to protect deep target tissue or structures and terminating the at least one pulse of cryogenic material prior to delivering treatment energy;
C. Delivering the pulsed energy to the target tissue or structures with a delivery system; and
D. Irradiating the target tissue or structures with the pulsed energy to cause selective thermally mediated treatment of the target tissue or structures such that there is minimal absorption of energy in deeper and adjacent tissue or structures and selective absorption of energy in target tissue or structures, and such that deeper tissue or structures remains at a temperature below the threshold for damage.

2. The method of claim 1 in which the step of cooling the surface of the skin is performed between about 100 and about 1000 msec prior to delivery of the pulsed treatment energy.

3. The method of claim 1 in which the target tissue or structures is a superficial pigmented lesion and the pulsed energy has a wavelength in the range from between about 440 nm to about 520 nm.

4. The method of claim 1 in which the pulsed energy has a pulse duration of 1 msec to 500 msec.

5. The method of claim 1 in which the cooling pulse has a duration of approximately between about 5 msec and about 200 msec.

6. The method of claim 1 in which the delay time between the end of the cooling pulse and beginning of the treatment energy pulse is between approximately 0.1 sec to 5.0 seconds.

7. The method of claim 1 in which the optical energy is delivered at the rate of between about 1 Joule/cm2 and about 100 Joules /cm2.

8. The method of claim 1 in which the target is a superficial vascular lesion.

9. The method of claim 8 in which the pulsed energy has a wavelength of between about 520 and about 600 nm, thereby preferentially selecting absorption of the pulsed energy by blood rather than melanin.

10. A treatment device that includes:
a treatment energy source capable of delivering optical energy with wavelength regions that are selectively absorbed by the target lesions and is highly scattered by the skin, the energy delivered in one or more short pulses between about 0.1 to 50.0 msec each thereby limiting penetration of the energy into the skin; and
a pulsed cooling system capable of delivering cooling to the skin between about 0.1 to 5.0 second prior to delivery of the treatment energy and capable of terminating cooling between about 0.1 and about 5.0 sec prior to delivery of the treatment energy.

11. A method for selective, thermally-mediated treatment of superficial pigmented lesions which includes the following steps:
Delivering cooling to the skin for a period of time between about 10 msec and about 50 msec to limit cooling to deeper structures;
Interrupting cooling of the skin for a period of time between about 100 msec and about 5000 msec; and
Delivering optical energy having one or more wavelengths between about 440 nm and about 520 nm to the target superficial pigmented lesions

12. A method for treatment of selective, thermally-mediated vascular lesions such as Rosacea which includes the following steps:
Delivering cooling to the skin for a period of time between about 5.0 msec and about 200.0 msec;
Interrupting cooling of the skin for a period of time between about 100 msec and about 5000 msec; and
Delivering optical energy having a wavelength between about 520 nm and about 600 nm to the target vascular lesions.
